(19) United States
(12) Patent Application Publication
(10) Pub. No.: US 2012/0071831 A1
(43) Pub. Date: Mar. 22, 2012

(54) NEEDLE-BASED MEDICAL DEVICE WITH NEEDLE SHIELD

(75) Inventor: Timothy J. Erskine, Sister Bay, WI (US)

(73) Assignee: ERSKINE MEDICAL LLC, High Falls, NY (US)

(21) Appl. No.: 13/259,715

(22) PCT Filed: Mar. 25, 2009
(86) PCT No.: PCT/US09/38246

§ 371 Date: Dec. 2, 2011

(51) Int. Cl. A61M 5/32

(52) U.S. Cl. 604/164.08

(57) ABSTRACT

A needle-based medical device including a needle shield assembly and a method for constructing the same are disclosed. The needle shield assembly may be deployed such that the device shields the needle passively on removal. A needle shield assembly includes a shield including a restraint member at a proximal end for engagement by an actuator in a non-shielding position. The needle shield assembly includes a shield having a stepped end cap at its distal end; a carrier positioned at the distal end of the shield that supports a needle blocking object abutting a first step of the end cap in the non-shielding position; and a biasing member within the shield for applying an axial force to the shield. Movement of the actuator disengages the restraint member such that the shield moves, by exertion of the biasing member, from the non-shielding position to a shielding position.

### TECHNICAL FIELD

The disclosure relates generally to needle based medical devices, and more particularly, to a needle based medical device with a needle shield.

### BACKGROUND ART

Blood collection devices and infusion sets are used very frequently in the medical industry. Since such devices rely on needles to puncture the skin and blood vessels they present a risk of needle sticks for healthcare professionals and patients. Needles may become contaminated with pathogens and if a contaminated needle is not properly handled after use, the chances of spreading diseases greatly increase. In order to prevent this, the sharp end of the needle should be quickly and securely shielded to prevent post-use needle sticks. Needle shields exist in the prior art which the user slides over the needle after use, but they do not shield the needle automatically on removal of the needle from the patient.

### SUMMARY OF THE INVENTION

One aspect of the invention includes a medical device such as a winged infusion set or winged needle. The device has a housing which has a needle fixed relative to it. The needle has a sharp distal end and a needle axis. A needle shield assembly is provided which has components including a needle shield, having proximal and distal ends. The needle shield is moveable relative to the housing, coaxial with the needle axis. A biasing member, preferably a coaxial coil spring, provides a biasing force to the needle shield, such that the biasing force can move the needle shield parallel to the needle axis in a distal direction relative to the housing. An actuator, preferably a push button, selectively restrains the needle shield when the needle shield is in a non-shielding position and releases the need shield so that the needle shield can move from the non-shielding position to a shielding position in which the needle shield covers the sharp distal end. The actuator can be operated prior to removal of the needle from a blood vessel, thus ensuring that the needle sharp distal end of the needle is shielded on removal of the needle from the blood vessel. A needle blocking object, preferably a ball bearing, is provided. It is moveable from a non-blocking position, when the needle shield is in the non-shielding position, to a blocking position in which the blocking object prevents the sharp distal end from exiting the needle shield, when the needle shield is in the shielding position. A carrier carries the needle blocking object when the needle shield moves from the non-shielding position to the shielding position and maintains the needle blocking object in the blocking position when the needle shield is in the shielding position. A lock is provided between the needle shield and the housing for preventing distal movement of the needle shield when the needle shield is in the shielding position. The distal end of the needle shield is provided with an inner surface such that when the needle shield is in the shielding position, the carrier maintains the needle blocking object against the inner surface. The inner surface comprises one or more steps. When the needle shield is in the non-shielding position, the needle blocking object is maintained substantially against one of the steps. In the shielding position, the needle blocking object may be maintained against another one of the steps.

Another aspect of the invention includes a method of shielding the tip of a needle for use with a needle based medical device. The method includes the steps of providing a housing; providing a needle shield having a proximal end and a distal end, the needle shield being moveable relative to the housing, and coaxial with the needle axis, from a non-shielding position into a shielding position in which the sharp distal tip is covered; providing a biasing member to exert a biasing force on the needle shield, such that the biasing force can move the needle shield parallel to the needle axis in a distal direction relative to the housing; providing an actuator for selectively restraining the needle shield when the needle shield is in a non-shielding position and releasing the needle shield; providing a needle blocking object and a carrier with a nest for the needle blocking object, such that the needle blocking object is moveable from a non-blocking position, when the needle shield is in the non-shielding position, to a blocking position in which the blocking object prevents the sharp tip from exiting the needle shield, when the needle shield is in the shielding position; inserting the sharp distal tip into a patient; actuating the actuator so that the needle shield is released; and removing the sharp distal tip from the patient, such that the needle shield moves, with the biasing force, from the non-shielding position to the shielding position. The actuating step can be performed prior to the step of removing, so that the needle shield is deployed passively.

These and other features of the disclosure will be more readily understood from the following detailed description of the various aspects of the invention.

### BRIEF DESCRIPTION OF THE DRAWINGS

FIG. 1 is a perspective view of the needle-based medical device of the preferred embodiment of the invention.

FIG. 2 is a cross-sectional view of the preferred embodiment of the needle-based medical device in a non-shielding position.

FIG. 3 is a cross-sectional view of the stepped end cap of the shield of the preferred embodiment of the needle-based medical device.

FIG. 4 is a perspective view of part of the shield when it is engaged by the shield actuator when the device is in the non-shielding position.

FIG. 5 is a perspective view of part of the shield when it is disengaged by the shield actuator just as the shield begins to move from the non-shielding position to the shielding position.

FIG. 6A is a cross-sectional view of the preferred embodiment of the needle-based medical device in a shielding position, and FIG. 6B is an enlarged cross-sectional view of FIG. 6A at the end of the shield.

FIG. 7 is a cross-sectional view of the distal end of an alternative embodiment needle-based medical device in the non-shielding position.

FIG. 8 is a cross-sectional view of the distal end of an alternative embodiment of the needle-based medical device in the shielding position.

FIG. 9 is a perspective view of the distal end of an alternative embodiment of the invention showing the carrier, the needle blocking object, and the needle.

FIGS. 10-16 are perspective views of the invention illustrating a preferred method of constructing the needle-based medical device.

It is noted that the drawings are not to scale. The drawings should not be considered as limiting the scope of the invention. Like numbering represents like elements in the drawings and the Detailed Description of the Invention.

### DETAILED DESCRIPTION OF THE INVENTION

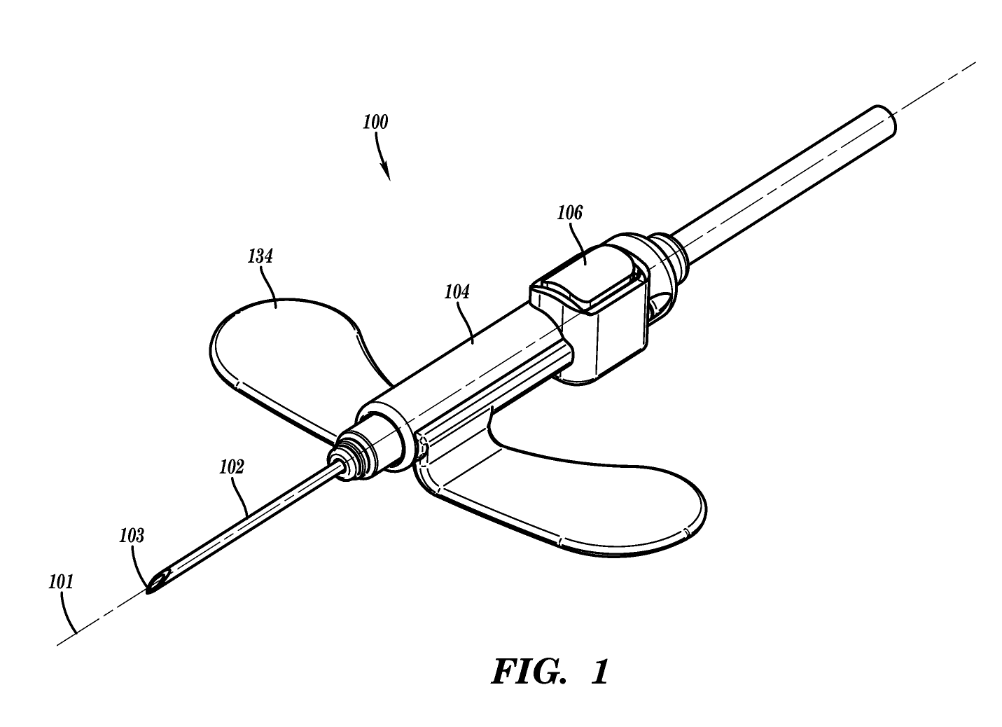

Referring now to FIG. 1, a needle-based medical device 100 is shown. Needle-based medical device 100 includes a needle 102 with a sharp distal end or tip 103 and a longitudinal axis 101, a housing 104 surrounding at least a part of needle 102, and an actuator 106 positioned in housing 104. Needle 102 is fixed relative to housing 104. Needle-based medical device 100 may include at least one outwardly extending wing 134. The purpose of wing 134 is to permit the device to be grasped between the finger and thumb of a user and to permit it to be taped to the patient. In the embodiment shown, housing 104 and wing 134 are formed as a single part. Wing 134 may alternatively be a separate part from housing 104, and the two parts attached as an assembly. This would allow each part to be formed of a different material for example to provide for soft, flexible wings and a more rigid housing, or to aid in assembly part handling. For example, the housing could be made out of polypropylene and the wing out of Thermoplastic Elastomer (TPE). In the embodiment shown, needle-based medical device 100 is illustrated as a blood collection device. However, this is not intended to limit the scope of the invention and it is understood that this invention may apply to any now known or later developed needle-based medical devices, in particular winged infusion sets.

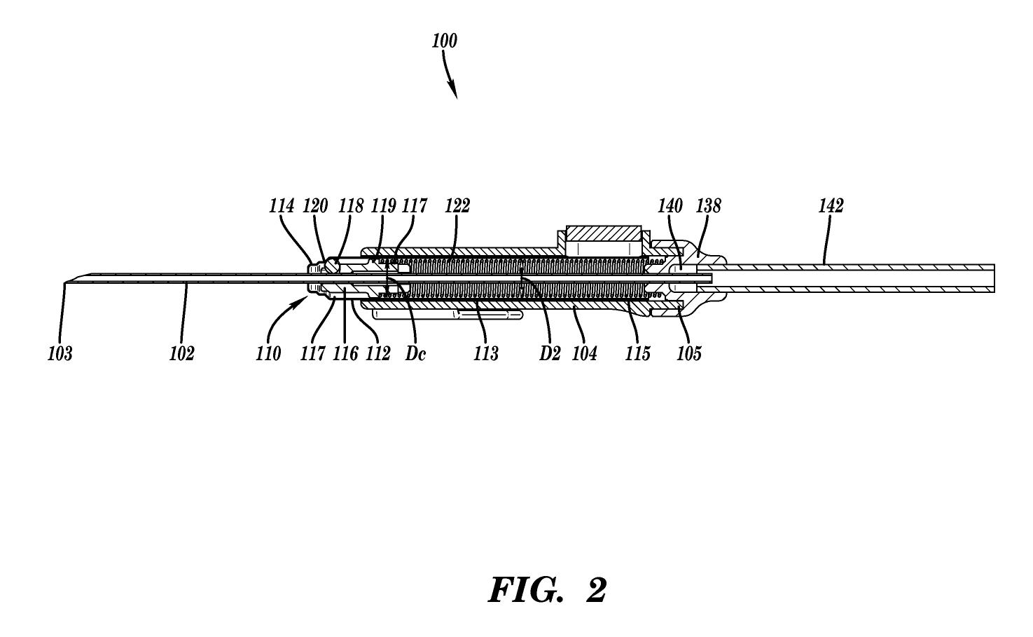

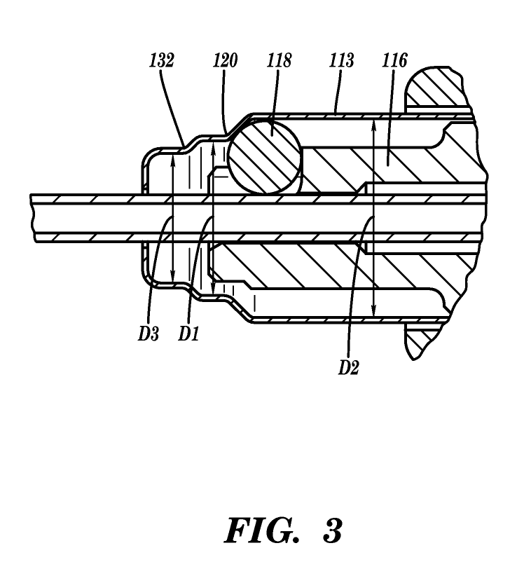

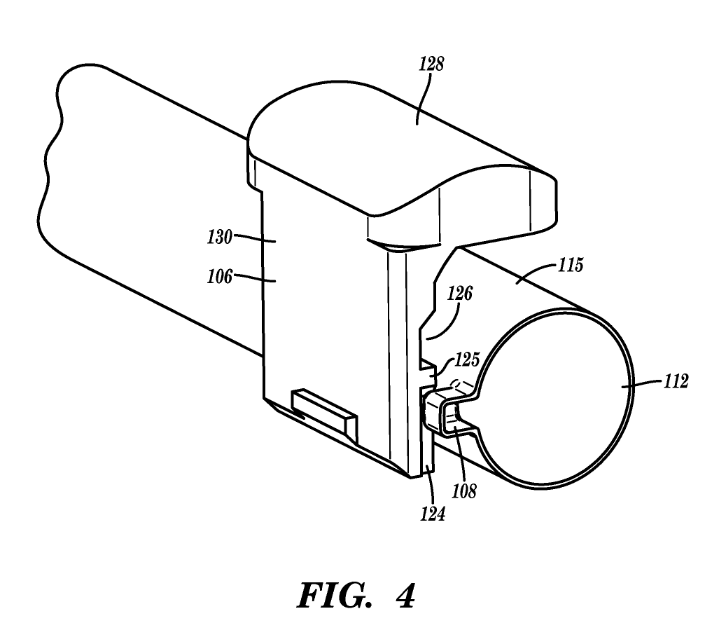

Referring now to FIGS. 2 and 3, cross-sectional views of needle-based medical device 100 will now be described. Needle-based medical device 100 has a needle shield assembly 110 positioned at least partially within housing 104 and coaxial with needle axis 101. Needle shield assembly 110 includes a shield 112 having a body 113 with a proximal end 115 and a distal end 117, and is moveable relative to housing 104. As will be described in greater detail herein, a restraint member 108 (FIG. 4) is positioned at proximal end 115 of body 113 to interact with actuator 106 when shield 112 is not covering needle tip 103, referred to here as the non-shielding position, as shown in FIG. 2. Shield 112 has a stepped end cap 114 at distal end 117. As shown in FIG. 3, stepped end cap 114 owes its stepped structure to three inner diameters, D1, D2 and D3. First step 120 (FIG. 3) lies between inner diameters D1 and D2. D2 is larger than first inner diameter D1 and the region of end cap 114 which has inner diameter D1 is distal of the region that has diameter D2. Inner diameters D2 is substantially uniform for the length of body 113. A second step 132 lies between inner diameters D1 and D3. Inner diameter D3 is less than D1 and the region of end cap 114 with inner diameter D3 distal of the region that has diameter D1. Although first step 120 and second step 132 are shown as circumferential, first step 120 and second step 132 may be formed as ribs, that is to say, they may be partially circumferential. Shield 112 may be a deep drawn thin walled metal tube and may be formed using standard transfer stamping production methods at very low costs. Shield 112 may alternatively be comprised of more than one part joined together using common assembly methods such as crimping, snap-fitting or adhesive bonding. For example, a tip can be attached to a tube to form a shield that functions similarly to a one-piece shield.

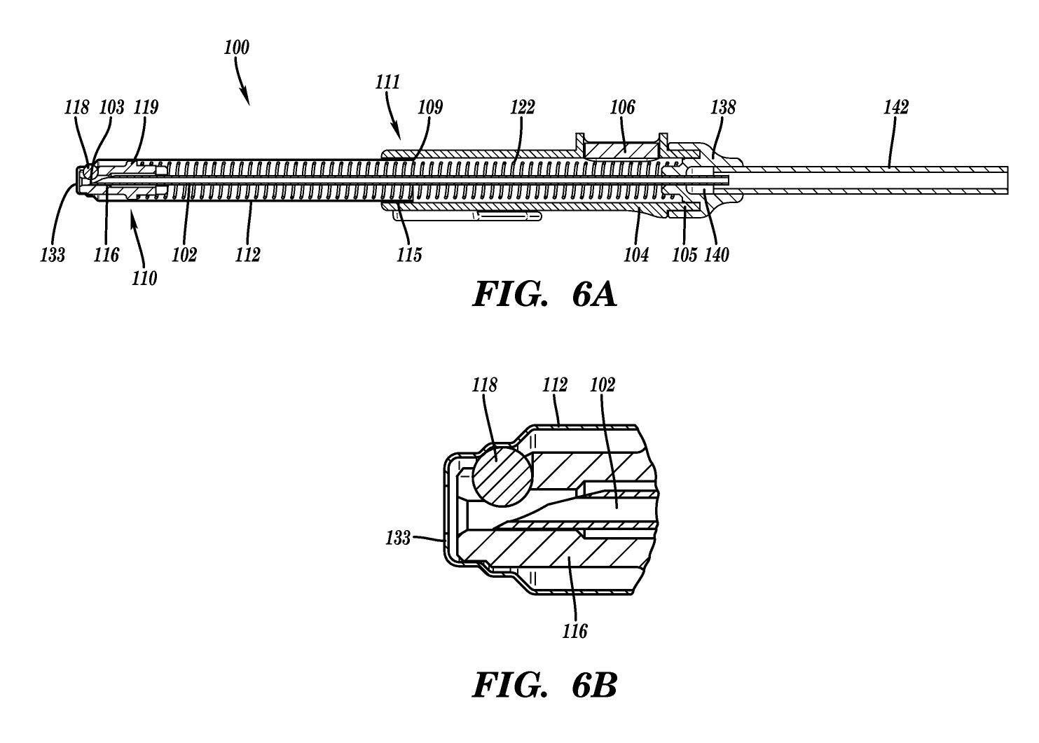

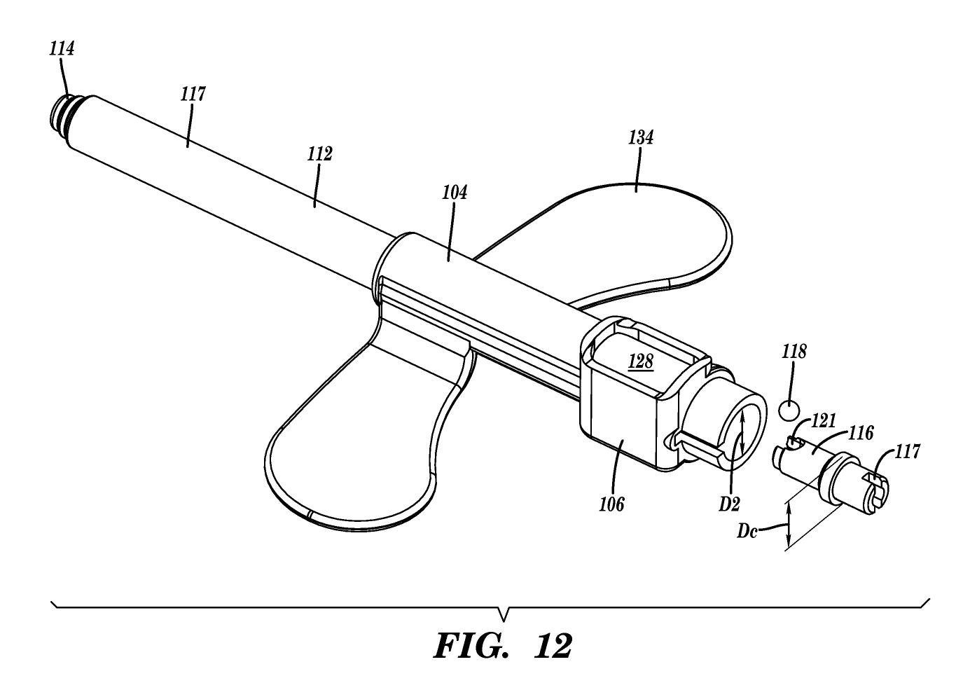

Returning to FIG. 2, needle shield assembly 110 also includes a carrier 116 positioned at distal end 117 of shield 112. Carrier 116 supports needle blocking object 118 in a nest 121 (see FIG. 12), while the device is in the non-shielding position and while it moves into the shielding position. Needle blocking object 118 is shown as a ball bearing; however, other structures may also be employed such as a cylindrical rod. Carrier 116 can be inexpensively manufactured by, for example, injection molding into a two-plate mold. Carrier 116 may include an enlarged portion 119 configured to position carrier coaxially within shield 112. Carrier 116 is positioned substantially within and coaxial with shield 112. When the device is in the shielding position, needle blocking object 118, is carried by carrier 116 in a position offset from needle axis 101. Further, as shown in FIG. 2, second inner diameter D2 of body 113 is substantially similar to an outer diameter Dc of carrier 116 (at portion 119), such that there is a close fit between carrier 116 and body 113. For example, there may be a 0.005 inch separation between an inner diameter of body 113 and outer diameter of carrier 116 (at portion 119). Carrier 116 preferably holds needle 102 concentric within shield 112 because the distal hole in shield 112 is large enough to accommodate a range of needle diameters so that only one shield configuration works for all gauge sizes. As best shown in FIG. 3, needle blocking object 118 is supported by carrier 116 such that it abuts first step 120 of stepped end cap 114 in the non-shielding position. Carrier 116 also maintains needle blocking object 118 in contact with needle 102 when shield 112 is in the non-blocking position (FIG. 2), and in the blocking position (FIG. 6A) when shield 112 is in the shielding position (FIG. 6A).

Again referring to FIG. 2, a biasing member 122 (e.g., a coil spring) is positioned within and coaxial with shield 112 through carrier 116 and blocking object 118. Biasing member 122 applies an axial biasing force to shield 112, which tends to push shield 112 in the distal direction, parallel to needle axis 101. Referring to FIG. 12, carrier 116 may include an end having a tool receiving member 117 such as a screw head slot for assembly purposes, i.e., such that a tool can insert carrier 116 into shield 104 at a certain orientation. Further, carrier 116 may be configured to have a close fit with needle 102 with a clearance of about 0.005 inches between the outside diameter of needle 102 and the inside diameter of carrier 116.

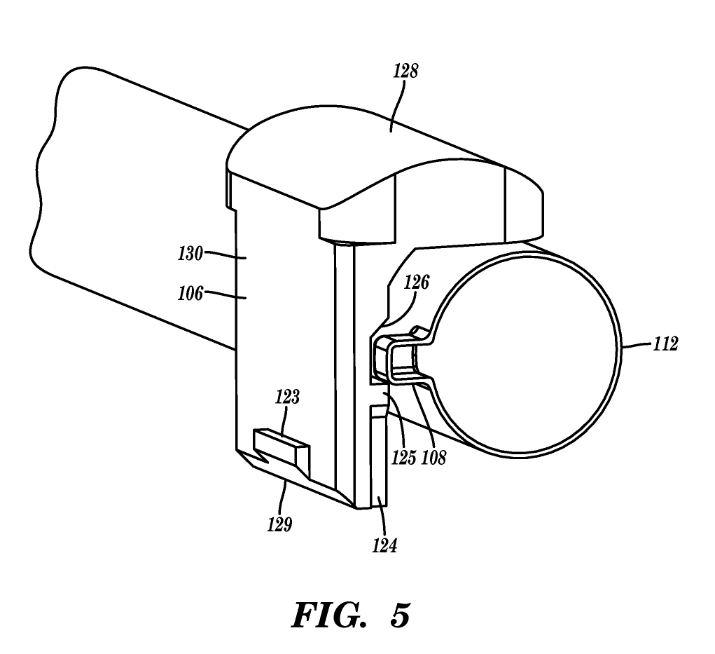

Referring to FIGS. 4 and 5, perspective views showing details of the interaction between actuator 106 and restraint member 108 of shield 112 are illustrated. The purpose of actuator 106 is to selectively restrain needle shield 112 against the biasing force of biasing member 122 when the needle shield is in the non-shielding position, and to release needle shield, allowing it to move from that position into the shielding position. FIG. 4 shows a perspective view of actuator 106 and restraint member 108 in the non-shielding position, and FIG. 5 shows actuator 106 and restraint member 108 just after actuation, i.e., as the shield just begins to move into the shielding position. Actuator 106 includes a first portion (or push button) 128 and a second portion 130. To actuate the device, i.e., move the shield from the non-shielding position to the shielding position, the user presses push button 128 with his or her thumb or finger. Second portion 130 includes a recess 124 that, as shown in FIG. 4, abuts restraint member 108 in the non-shielding position, thus maintaining shield 112 in the non-shielding position (FIG. 2) against the bias of biasing member 122. Second portion 130 also includes a channel 126. A detent 125 is provided between recess 124 and channel 126. Channel 126 lies substantially parallel to needle axis 101 and is configured to allow restraint member 108 to move along it, also substantially parallel to needle axis 101. Channel 126 is positioned such that when push button 128 of actuator 106 is depressed by the user along a line perpendicular to needle axis 101 (FIG. 1), as shown in FIG. 5, to a disengaged position, restraint member 108 can travel along channel 126 of actuator 106, thus releasing shield 112 and allowing distal movement of shield 112 into the shielding position. More particularly, when push button 128 is depressed, restraint member 108 moves up and over detent 125 between recess 124 and channel 126. This detent 125 provides a slight resistance during the depression of push button 128 (a “click”) for tactile feedback to the user and further serves to prevent actuator 106 from inadvertently moving and releasing shield 112 due to vibration or acceleration during shipping, manufacturing or handling.

Thus, when push button 128 is pressed and restraint member 108 is disengaged, shield 112 can move, due to the biasing force provided by biasing member 122, from the non-shielding position to the shielding position. During use, push button 128 may be pushed by the user either after needle 102 is withdrawn from the patient, or after the needle 102 has been inserted into the blood vessel but prior to withdrawal of needle 102 from the blood vessel. In the latter approach, once push button 128 has been pushed, the device operates as a passive device in that needle 102 cannot be removed from the patient without shield 112 extending to cover needle 102. This action occurs without further action by the user. That is to say, needle 102 is automatically or passively shielded as it is removed from the patient. Biasing member 122 is designed so that the biasing force applied to shield 112 is light enough to prevent the device from being pushed out of the patient's skin when used as a passive device by pushing push button 128 while needle 102 is still in the patient's blood vessel.

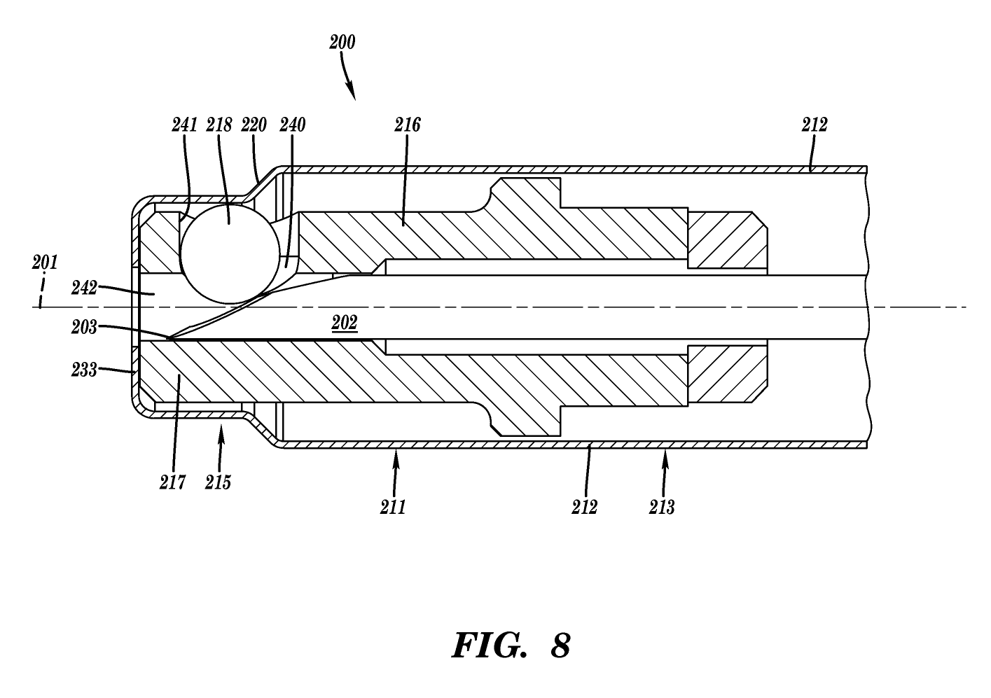

Returning to FIG. 3, it can be seen that in the non-shielding position, needle blocking object 118 abuts step 120 and touches needle 102. As shield 112 moves from the non-shielding position into the shielding position, needle blocking object 118 moves towards needle axis 101 and distally, from being in abutment with step 120 to being in abutment with step 132. Needle blocking object 118 is trapped by nest 121 (FIG. 12) of carrier 116 and step 132, offset from needle axis 101, thus preventing distal movement of needle tip 103. Needle tip 103 may protrude slightly beyond needle blocking object 118, but is enclosed within distal tip 114 of shield 112, which extends beyond the distal end of carrier 116. That is, step 132 is distanced from the distal end of shield 112 such that sharp distal end 103 of needle 102 cannot emerge from shield 112. Thus, in the shielding position, as shown in FIGS. 6A-B, shield 112 covers sharp distal end 103 of needle 102. Needle blocking object 118 is positioned to prevent proximal movement of shield 112 which would otherwise re-expose sharp distal end 103 of needle 102. As shown in FIG. 6A, a lock 111 is also provided between needle shield 112 and housing 104 for preventing distal movement of the needle shield when the needle shield is in the shielding position. In one embodiment, lock 111 includes a stop 109 (FIG. 6A) in housing 104 that is configured to abut restraint member 108 to prevent further distal movement of shield 112 in the shielding position, such that proximal end 115 of shield 112 is prevented from exiting housing 104. In the preferred embodiment, in the shielding position, carrier 116 is pressed against the proximal side of needle blocking object 118 by the biasing force of biasing member 122, and the distal end of carrier 116 fits closely with third inner diameter D3 of shield 112, but may not rest against the distal end of the shield. In an alternative embodiment, however, the distal end of carrier 116 may abut distal end 133 of shield 112, as shown in FIG. 8. Needle blocking object 118 is offset from needle axis 101 and shield 112 in the shielding position.

Referring now to both FIGS. 3 and 6B, when needle shield assembly 110 moves from the non-shielding position to the shielding position, needle blocking object 118 moves from a first position behind first step 120 to a second position behind second step 132. In both the first and second position, needle blocking object 118 is offset from needle axis 101.

Persons of ordinary skill in the art will recognize that where items are described as being in contact with or abutment with each other, materials may be interposed between them (for example, shims, lubricants, pads and coatings) or small gaps may exist without departing from the basic intent behind the interaction of the items.

As shown in FIGS. 2 and 6A, needle 102 is secured to a needle hub 138, which is attached to proximal end 105 of housing 104. Needle hub 138 may be attached to housing 104 in a variety of ways known in the art. These include, but are not limited to, a snap fit or an adhesive bond as well as the structure and method shown in co-pending application Ser. No. 11/536,236, entitled NEEDLE-BASED MEDICAL DEVICE INCLUDING NEEDLE GUIDE AND METHOD FOR CONSTRUCTING, which is incorporated herein by reference. Needle hub 138 may also include a glue well 140. Glue well 140 receives glue for securing the proximal end of needle 102 to needle hub 138. It may also attach an extension tube 142 to needle hub 138.

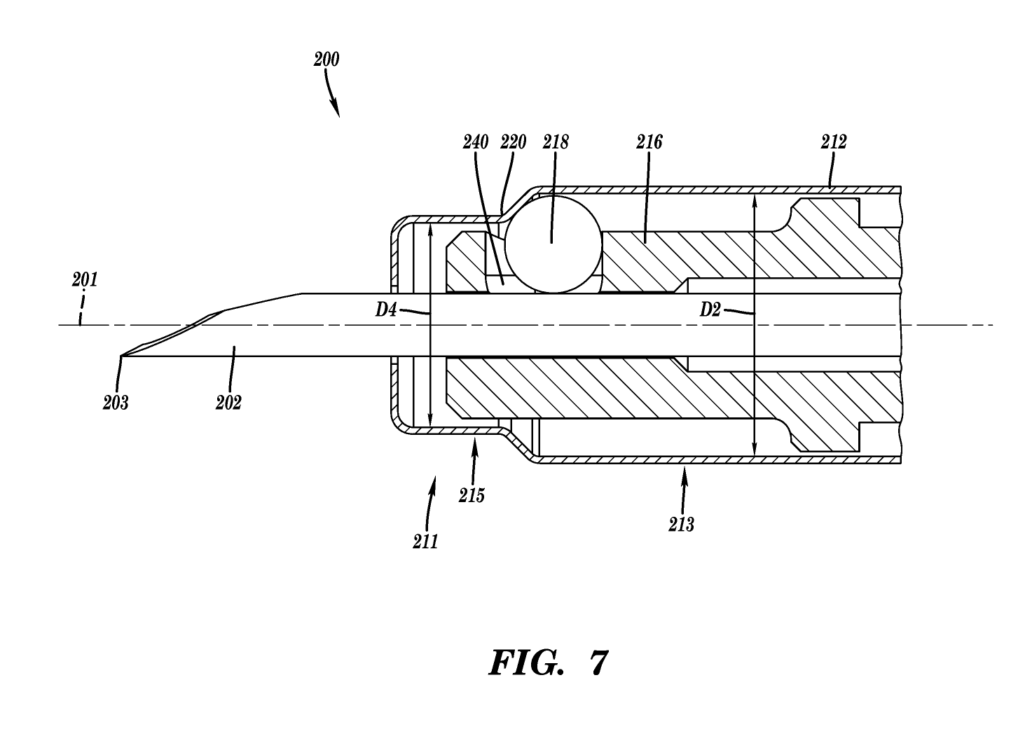

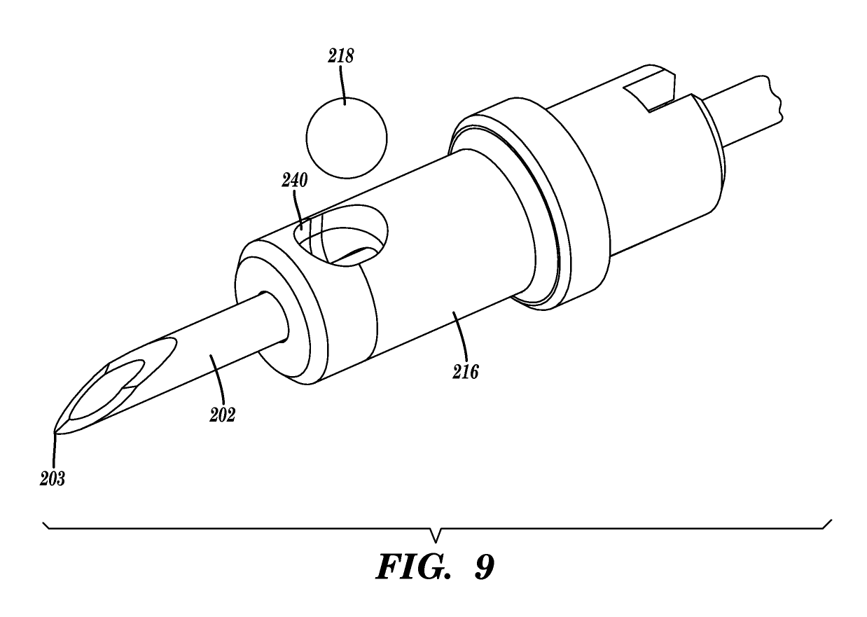

FIGS. 7-8 are cross-sectional views of an alternative embodiment in which the distal end 211 of shield 212 of needle-based medical device 200 has a single step 220. Referring to FIG. 7, in the non-shielding position, the top part of needle blocking object 218 abuts step 220. Step 220 is formed due to shield 212 having regions with two inner diameters. First inner diameter D4, which is towards distal end 211 of shield 212, is less than second inner diameter D2 of shield 212. A region 213 on shield 212 which has second inner diameter D2 is proximal of a region 215 with first inner diameter D4. Needle blocking object 218 is carried in nest 240 of carrier 216, touching needle 202 and abutting step 220. When shield 212 moves from the non-shielding position (shown in FIG. 7) to the shielding position (shown in FIG. 8), step 220 cams needle blocking object so that it moves slightly distally and radially towards needle axis 201. In the shielding position shown in FIG. 8, needle blocking object 218 abuts distal surface 241 of nest 240. Nest 240 supports needle blocking object 218 partially within a lumen 242, slightly offset from axis 201 of needle 202, thus blocking sharp distal end 203 of needle 202 and preventing distal movement of needle 202 out of shield 212. In this position, sharp distal end 203 protrudes slightly beyond needle blocking object 218, but since needle blocking object 218 is distanced from a distal end of shield 212, sharp distal end 203 cannot emerge from shield 212. Distal end 217 of carrier 216 extends slightly beyond sharp distal end 203 so that end 203 will not extend out of carrier 216. Distal end 217 of carrier 216 abuts distal end 233 of shield 212 in the shielding position. FIG. 9 shows an enlarged perspective view of carrier 216 including nest 240.

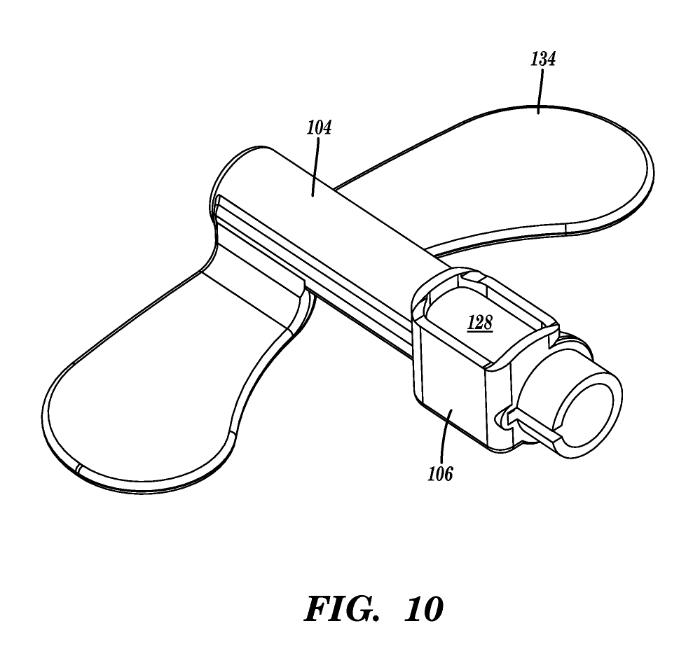

Referring now collectively to FIGS. 10-16, a method of constructing needle-based medical device 100 shown in FIGS. 2, 6A and 6B is described. The embodiment discussed herein includes first step 120 and second step 132, where first step 120 has first inner diameter D1 and second step 132 has third inner diameter D3 and diameter D3 is less than diameter D2. However, it is not intended to limit the method of constructing needle-based medical device 100 to this embodiment. The method of constructing may also apply to needle-based medical device 200 discussed above, with respect to FIGS. 7-8, which only includes single step 220.

As shown in FIG. 10, in a first step, housing 104 is provided, and actuator 106 is placed in housing 104 in a disengaged position (i.e., the position it will be in when needle shield 112 is in the shielding position). That is, first portion 128 of actuator 106 is depressed relative to housing 104.

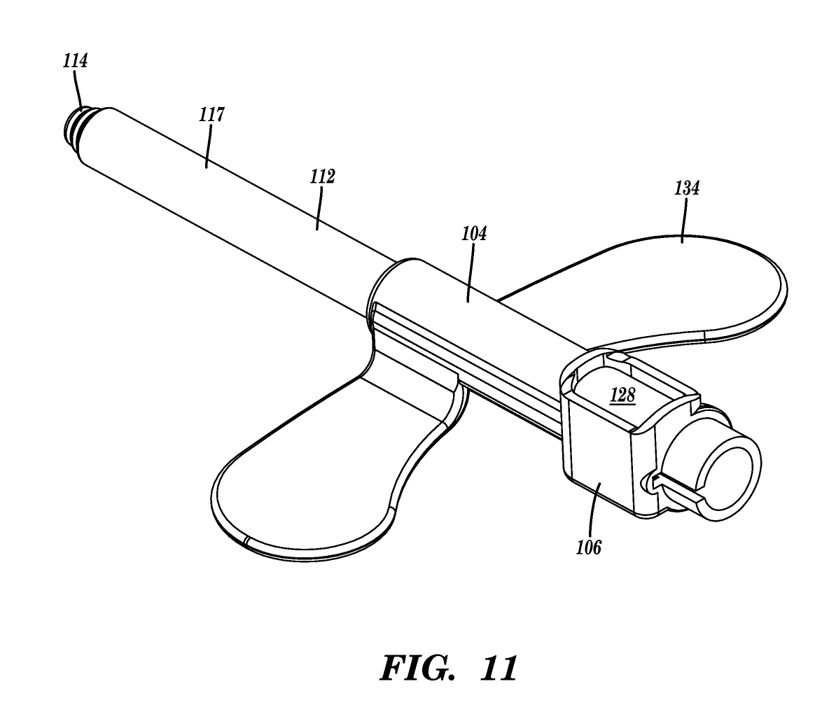

Next, referring to FIG. 11, shield 112 is inserted into housing 104 in the shielding position. As shown in FIG. 11, shielding position includes shield 112 fully extended from needle shield housing 104, but with the proximal end of shield 112 within housing 104.

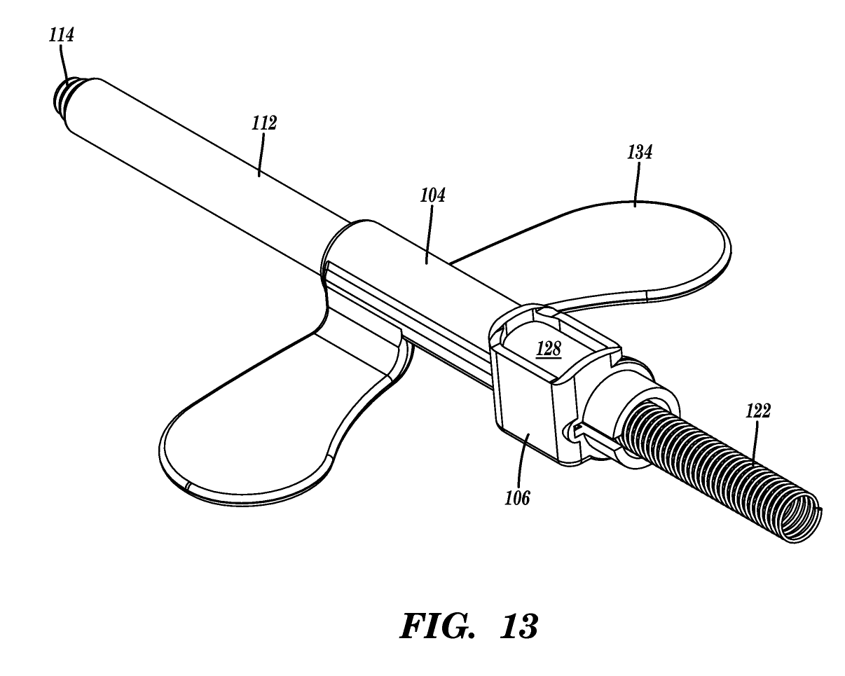

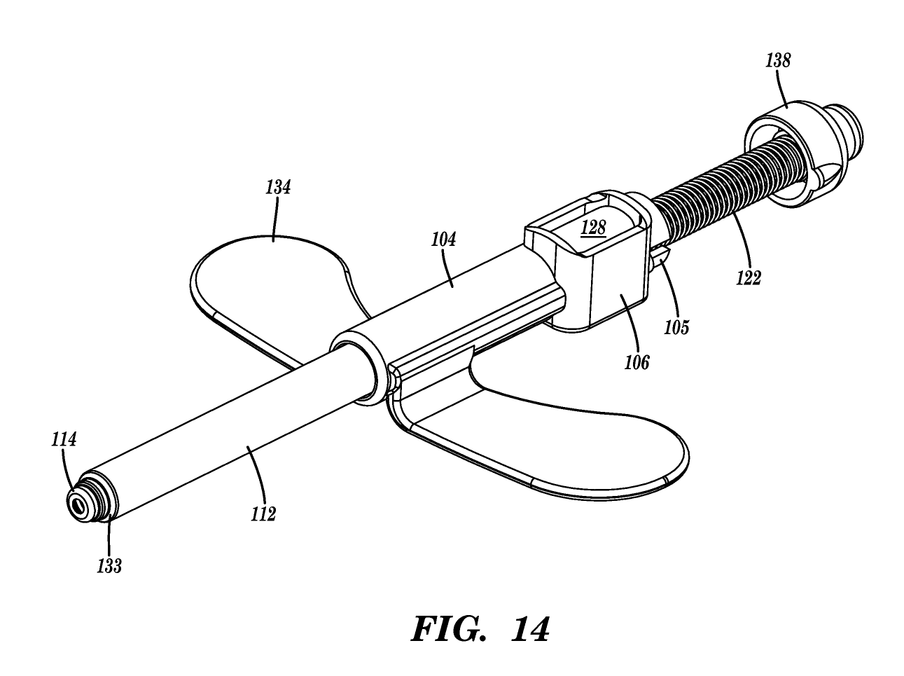

Referring now to FIG. 12, carrier 116 is inserted into proximal end 115 of shield 112 and positioned at distal end 117 of shield 112. Carrier 116 will abut distal end 117 of shield 112 and needle blocking object 118 will abut second step 132 of stepped end cap 114 (See FIG. 2). Carrier 116 has a diameter Dc that is substantially similar to second inner diameter D3 of body 113 of shield 112, so that there is a close fit. Referring to FIG. 13, biasing member 122 is inserted into shield 112 from proximal end 115. Next, as shown in FIG. 14, needle hub 138 is attached to proximal end 105 of housing 104. Needle hub 138 engages biasing member 122.

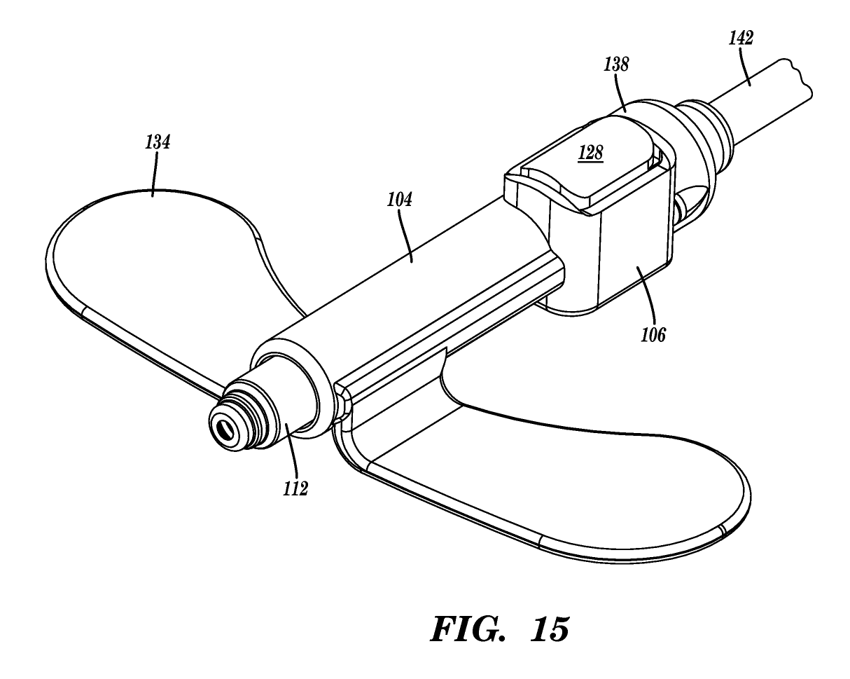

Referring now to FIG. 15, shield 112 is pushed back into housing 104 against the bias of biasing member 122. As shield 112 is depressed, actuator 106 is still depressed so that restraint member 108 of shield 112 slides through channel 126. When shield 112 is pushed far enough into housing 104, restraint member 108 is engaged by recess 124 of actuator 106 (See FIG. 4). This engagement may be accomplished by applying force to a retaining end 129 of actuator 106 such that first portion 128 is no longer depressed in the disengaged position, thus allowing restraint member 108 and actuator 106 to achieve the position shown in FIG. 4. Actuator 106 is retained in housing 104 by an actuator stop 123 (See FIG. 5).

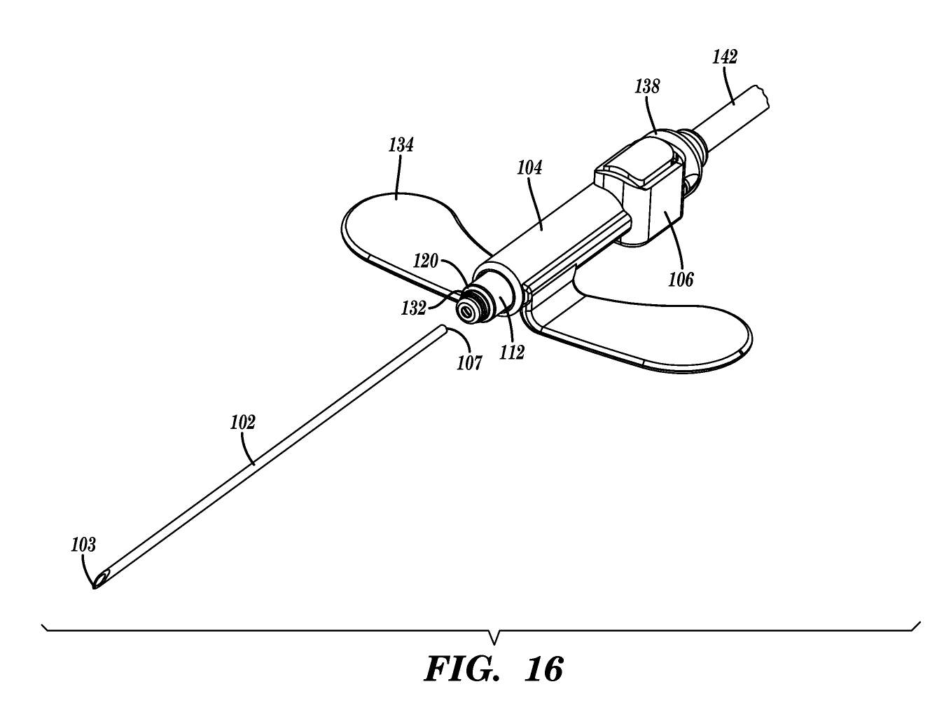

As shown next in FIG. 16, proximal end 107 of needle 102 is now positioned and inserted into shield 112 and carrier 116 such that needle blocking object 118 moves to axially abut first step 120 (See FIG. 3) into a non-shielding position. That is, proximal end 101 of needle 102 moves needle blocking object 118 out of the way by pushing needle blocking object 118 from second step 132 (if provided) to abut first step 120. Shield 112 and carrier 116 extend along a substantially similar axis. Next, proximal end 101 of needle 102 is secured to needle hub 138. This securing may include introducing glue into glue well 140 (FIG. 2) between needle hub 138 and proximal end 107 of needle 102. Glue well 140 may also attach extension tube 142 to needle hub 138.

The terms “first,” “second,” and the like, herein do not denote any order, quantity, or importance, but rather are used to distinguish one element from another, and the terms “a” and “an” herein do not denote a limitation of quantity, but rather denote the presence of at least one of the referenced item. The modifier “about” used in connection with a quantity is inclusive of the stated value and has the meaning dictated by the context, (e.g., includes the degree of error associated with measurement of the particular quantity). The suffix “(s)” as used herein is intended to include both the singular and the plural of the term that it modifies, thereby including one or more of that term (e.g., the metal(s) includes one or more metals).

The foregoing description of various aspects of the invention has been presented for purposes of illustration and description. It is not intended to be exhaustive or to limit the invention to the precise form disclosed, and obviously, many modifications and variations are possible. Such modifications and variations that may be apparent to an individual in the art are included within the scope of the invention as defined by the accompanying claims.

What is claimed is:

1. A medical device comprising:
a housing;
a needle fixed relative to the housing, the needle having a sharp tip and a needle axis;
a needle shield assembly comprising:
a needle shield having a proximal end and a distal end, the needle shield being moveable relative to the housing, and coaxial with the needle axis;
a biasing member providing a biasing force to the needle shield, such that the biasing force can move the needle shield parallel to the needle axis in a distal direction relative to the housing;
an actuator for selectively restraining the needle shield when the needle shield is in a non-shielding position and releasing the needle shield so that it can move from the non-shielding position to a shielding position in which the needle shield covers the sharp tip;
a needle blocking object moveable from a non-blocking position, when the needle shield is in the non-shielding position, to a blocking position in which the blocking object prevents the sharp tip from exiting the needle shield, when the needle shield is in the shielding position;
a carrier having a nest for carrying the needle blocking object when the needle shield moves from the non-shielding position to the shielding position and for maintaining the needle blocking object in contact with the needle when the needle shield is in the non-shielding position and in the blocking position when the needle shield is in the shielding position.

2. The medical device of claim 1 further comprising a lock between the needle shield and the housing for preventing distal movement of the needle shield when the needle shield is in the shielding position.

3. The medical device of claim 1 wherein the biasing member is a coil spring, coaxial with the needle axis.

4. The medical device of claim 1 wherein the needle blocking object is a ball bearing.

5. The medical device of claim 1 wherein the distal end of the needle shield comprises an inner surface such that when the needle shield is in the shielding position, the carrier maintains the needle blocking object against the inner surface.

6. The medical device of claim 5 wherein the inner surface comprises one or more steps.

7. The medical device of claim 6 wherein, when the needle shield is in the non-shielding position, the needle blocking object is maintained against one of said steps.

8. The medical device of claim 6 wherein, when the needle shield is in the shielding position, the needle blocking object is maintained against one of said steps.

9. The medical device of claim 1 wherein the distal end of the needle shield comprises an inner surface and when the needle shield is in the shielding position, part of the carrier abuts the inner surface.

10. The medical device of claim 9 wherein, when the needle shield is in the shielding position, the needle blocking object lies between part of the carrier and the inner surface.

11. The medical device of claim 1 further comprising at least one winged member extending outwardly from the housing.

12. A medical device comprising:
a housing;
a needle fixed relative to the housing, the needle having a sharp tip and a needle axis;
a needle shield assembly comprising:
a needle shield having a proximal end and a distal end, the needle shield being moveable relative to the housing, and coaxial with the needle axis, the distal end of the needle shield comprising one or more steps;
a biasing member providing a biasing force to the needle shield, such that the biasing force can move the needle shield parallel to the needle axis in a distal direction relative to the housing;
an actuator for selectively restraining the needle shield when the needle shield is in a non-shielding position and releasing the needle shield so that the needle shield can move from the non-shielding position to a shielding position in which the needle shield covers the sharp tip;
a needle blocking object moveable from a non-blocking position, when the needle shield is in the non-shielding position, to a blocking position in which the blocking object prevents the sharp tip from exiting the needle shield, when the needle shield is in the shielding position;
a carrier having a nest for carrying the needle blocking object when the needle shield moves from the non-shielding position to the shielding position and for maintaining the needle blocking object in contact with the needle when the needle shield is in the non-shielding position and in the blocking position when the needle shield is in the shielding position;
and wherein, when the needle shield is in a non-shielding position, the needle blocking object is maintained against one of said steps.

13. The medical device of claim 12 wherein, when the needle shield is in the shielding position, the needle blocking object is maintained against one of said steps.

14. The medical device of claim 12 wherein the distal end of the needle shield comprises an inner surface and when the needle shield is in the shielding position, part of the carrier abuts the inner surface.

15. The medical device of claim 12 wherein, when the needle shield is in the shielding position, the needle blocking object lies between part of the carrier and the inner surface.

16. The medical device of claim 1 further comprising at least one winged member extending outwardly from the housing.

17. The medical device of claim 12 further comprising a lock between the needle shield and the housing for preventing distal movement of the needle shield when the needle shield is in the shielding position.

18. The medical device of claim 12 wherein the biasing member is a coil spring, coaxial with the needle axis.

19. The medical device of claim 12 wherein the needle blocking object is a ball bearing.

20. For use in a needle based medical device having a needle with an axis and a sharp distal tip, a method of shielding the sharp distal tip comprising the steps of:
providing a housing;
providing a needle shield having a proximal end and a distal end, the needle shield being moveable relative to the housing, and coaxial with the needle axis, from a non-shielding position into a shielding position in which the sharp distal tip is covered;
providing a biasing member exerting a biasing force on the needle shield, such that the biasing force can move the needle shield parallel to the needle axis in a distal direction relative to the housing;
providing an actuator for selectively restraining the needle shield when the needle shield is in a non-shielding position and releasing the needle shield;
providing a needle blocking object and a carrier with a nest for the needle blocking object, such that the needle blocking object is moveable from a non-blocking position, when the needle shield is in the non-shielding position, to a blocking position in which the blocking object prevents the sharp tip from exiting the needle shield, when the needle shield is in the shielding position;
inserting the sharp distal tip into a patient;
actuating the actuator so that the needle shield is released;
removing the sharp distal tip from the patient, such that the needle shield moves, with the biasing force, from the non-shielding position to the shielding position.

21. The method of claim 20 wherein the step of actuating is performed prior to the step of removing.
